(19) United States
(12) Patent Application Publication
(10) Pub. No.: US 2010/0048908 A1
(43) Pub. Date: Feb. 25, 2010

(54) Process for Remifentanil Synthesis

(76) Inventors: Brian Kai-Ming Cheng, Chesterfield, MO (US); Robert E. Halvachs, Belleville, IL (US)

Correspondence Address: Christine R Cooke;Mallinckrodt Inc, 675 McDonnell Blvd, P O Box 5840, St Louis, MO, 63134, US

(21) Appl. No.: 12/515,958

(22) PCT Filed: Nov. 13, 2007
(86) PCT No.: PCT/US2007/023962

§ 371 Date: May 22, 2009

Related U.S. Application Data
(60) Provisional application No. 60/861,717, filed on Nov. 29, 2006.

(51) Int. Cl. C07D 211/58

(52) U.S. Cl. 546/224

(57) ABSTRACT

A process for synthesizing remifentanil or carfentanil, as well as intermediates for use in the preparation of a synthetic opiate or opioid compound by alkylating a substituted 4-piperidine in the presence of a base to form an intermediate that is further alkylated with an electrophilic alkylating agent and acylated to produce the compound.

### FIELD OF THE INVENTION

The present invention generally relates to a process for synthesizing opiate or opioid analgesics and anesthetics, and precursors thereof. In particular, the present invention relates to a process for synthesizing opiate or opioid compounds such as, for example, remifentanil, carfentanil, sufentanil, fentanyl, and alfentanil. In particular, the present invention relates to a preparation process with fewer steps, faster reaction time, reduced costs, improved safety, and higher efficiency than processes known in the art for producing remifentanil and carfentanil.

### BACKGROUND OF THE INVENTION

Analgesics, such as remifentanil and carfentanil, have been prepared in synthetic processes comprising six and seven steps. Examples of such processes are outlined in U.S. Pat. Nos. 5,106,983 and 5,019,583. However, these syntheses often require protection and deprotection steps of reactive moieties, resulting in increased process costs due to reduced production efficiency and additional material costs. These processes typically use cyanide compounds, which substantially increase safety and environmental concerns, waste disposal cost, and require EPA registration.

A process that does not use cyanide compounds would improve safety, reduce cost, and eliminate the need for EPA registration. In addition, a process with fewer synthetic steps and faster reaction times would improve process efficiencies and reduce the overall cost of synthesizing analgesics and anesthetics.

### SUMMARY OF THE INVENTION

Among the several features of the present invention, therefore, can be noted the provision of a process for synthesizing intermediates and final synthetic opiate or opioid compounds such as, for example, remifentanil, carfentanil, sufentanil, fentanyl, and alfentanil; the provision of preparing an analgesic or anesthetic; the provision of a process that requires relatively fewer steps for synthesizing remifentanil; the provision of a process that allows for decreased overall reaction time; the provision of a process that requires relatively fewer steps for synthesizing carfentanil; and the provision of such a process wherein remifentanil is prepared from a substituted 4-pipeddine.

Briefly, therefore, the present invention is directed to a process for the preparation of an analgesic or anesthetic. Specifically, the process comprises alkylating with a first alkylating agent a compound (I) having the formula:

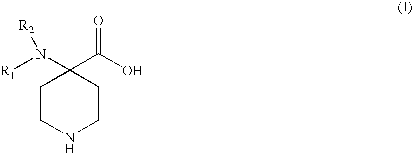

wherein R₁ and R₂ are independently hydrogen, hydrocarbyl or substituted hydrocarbyl in the presence of a solvent and a base to form intermediate compound (II):

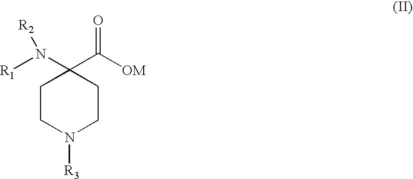

wherein M is hydrogen, a cation, amino, or substituted amino and R₃ is hydrocarbyl or substituted hydrocarbyl; alkylating the compound (II) with a second alkylating agent to form an intermediate compound (III):

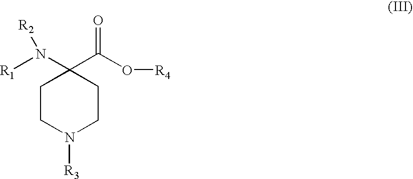

wherein R₄ is hydrocarbyl or substituted hydrocarbyl; and

acylating the compound (III) with an acylating agent in the presence of a solvent to form the opiate or opioid, compound (IV):

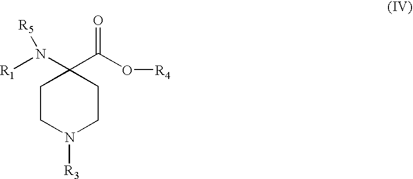

wherein R₅ is —C(O)—R₆ and R₆ is hydrocarbyl or substituted hydrocarbyl.

In some aspects of this invention, intermediate compound (II) is isolated, while in other aspects, intermediate compound (II) is not isolated.

Other aspects and features of this invention will be in part apparent and in part pointed out hereinafter.

### DETAILED DESCRIPTION

In accordance with the present invention, an improved process for synthesizing analgesics or anesthetics has been discovered. The improved process reduces the number of process steps required to synthesize the analgesics or anesthetics, decreases the overall reaction time, and avoids the use of cyanide compounds. The process also improves the yield of the synthesized analgesic or anesthetic product as compared to processes known in the art.

In one embodiment, the process of the present invention results in the synthesis of a compound having the formula (IV):

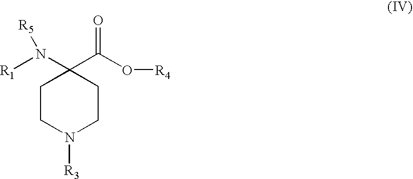

wherein R₅ is C(O)R₆, R₁ is hydrogen, hydrocarbyl, or substituted hydrocarbyl, and R₃ and R₄ are independently hydrocarbyl or substituted hydrocarbyl.

In one embodiment, the present invention can be used to synthesize remifentanil, chemically identified as 3-[4-methoxycarbonyl-4-[(1-oxopropyl) phenylamino]-1-piperidine]propanoic acid methyl ester, having the formula (V):

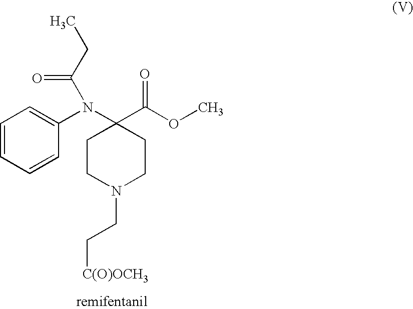

In another embodiment, the present invention can be used to synthesize carfentanil, chemically identified as 4((1-oxopropyl)phenylamino)-1-(2-phenylethyl)-4-pipeddinecarboxylic acid, methyl ester, having the formula (VI):

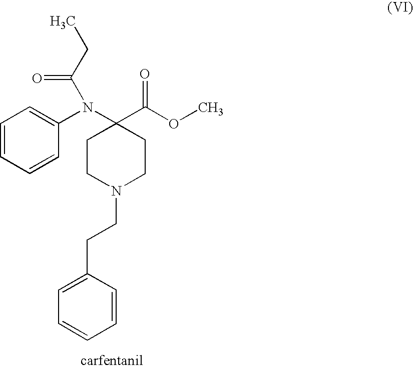

The new process of the present invention for synthesizing opiate oropioid analgesics and anesthetics includes the synthesis of a series of intermediates, each of which may be used in the preparation of synthetic opiate or opioid compounds. Scheme 1, below, illustrates a first step in the process wherein a substituted 4-piperidine, compound (I), is reacted with a first alkylating agent and a base in an inert solvent to form intermediate compound (II).

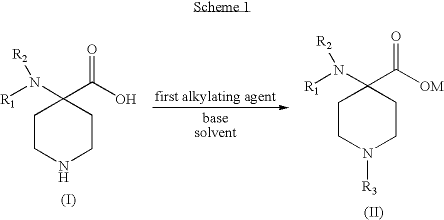

In Scheme 1, compound (I) is mixed with a first alkylating agent in the presence of a solvent and a base to form intermediate compound (II), wherein R₁ and R₂ are independently hydrogen, hydrocarbyl, or substituted hydrocarbyl, R₃ is hydrocarbyl or substituted hydrocarbyl, and M is hydrogen, a cation, amino or substituted amino.

In one embodiment, R₁ and R₂ are independently selected from the group consisting of hydrogen, aryl, substituted aryl, C₁₋₁₈alkyl, cycloalkyl, substituted cycloalkyl, heterocyclic, R₇OR₈—, and R₉R₈—, wherein R₇ is hydrocarbyl or substituted hydrocarbyl, R₈ is hydrocarbylene or substituted hydrocarbylene and R₉ is selected from the group consisting of cycloalkyl, substituted cycloalkyl, and heterocyclic. In one example of this embodiment, R₇ is substituted or unsubstituted alkyl, alkoxy, alkenyl, alkenyloxy, or aryl, R₈ is substituted or unsubstituted alkylene, alkyleneoxy, alkenylene, alkenyleneoxy, or arylene, and R₉ is C₃₋₆cycloalkyl, substituted C₃₋₆cycloalkyl, or a 5- to 7-membered heterocyclic comprising 1 to 5 heteroatoms selected from oxygen, sulfur, and nitrogen. In one preferred embodiment, R₁ and R₂ are independently hydrogen, substituted or unsubstituted alkyl, alkoxy, or aryl. In another preferred embodiment, R₁ and R₂ are independently selected from hydrogen, C₁₋₈alkyl, and phenyl and more preferably R₁ is phenyl and R₂ is hydrogen.

Typically, R₃ is selected from the group consisting of aryl, substituted aryl, aralkyl, C₁₋₁₈alkyl, R₁₀OC(O)R₁₁—, R₁₀C(O)OR₁₁ —, R₁₀OR₁₂OC(O)R₁₁—, R₁₃R₁₁—, and R₁₄R₁₁—, wherein R₁₀ is hydrocarbyl or substituted hydrocarbyl, R₁₁ and R₁₂ are independently hydrocarbylene or substituted hydrocarbylene, R₁₃ is cycloalkyl or substituted cycloalkyl, and R₁₄ is heterocyclic. In one example of this embodiment, R₁₀ is alkyl, alkoxy, alkenyl, aryl, aralkyl, or alkenyloxy, R₁₁ and R₁₂ are independently alkylene, alkyleneoxy, alkenylene, arylene, aralkylene, or alkenyleneoxy, R₁₃ is C₅₋₇ cycloalkyl, and R₁₄ is a 5- to 7-membered heterocyclic. In another example of this embodiment, R₁₀ is linear or branched alkyl, alkoxy, alkenyl, or alkenyloxy having about 1 to about 18 carbon atoms or an aryl or aralkyl, R₁₁ and R₁₂ are independently linear or branched alkylene, alkyleneoxy, alkenylene, or alkenyleneoxy having about 1 to about 18 carbon atoms or an arylene or aralkylene, R₁₃ is C₅₋₇ cycloalkyl, and R₁₄ is a 5- to 7-membered heterocyclic comprising 1 to 5 heteroatoms selected from oxygen, sulfur, and nitrogen. In a preferred embodiment, R₃ is benzyl, substituted benzyl, phenyl, substituted phenyl (e.g., 2-phenylethyl), methyl propionyl, ethyl propionyl, 2-(2-thienyl)ethyl, or 2-(4-ethyl-4,5-dihydro-5-oxo-1H-tetrazol-1-yl)ethyl.

Typically, M is hydrogen or an alkali or alkaline earth metal cation. In one example of this embodiment, M is hydrogen or a sodium, potassium, or lithium cation. In a preferred embodiment, M is tetraalkylamino.

General examples of alkylating agents include compounds having the structure:

L-R₁₅—R₁₆

wherein L is a displacement or leaving group. In one embodiment, L and R₁₆ are independently hydrocarbyl or substituted hydrocarbyl, and R₁₅ is hydrocarbylene or substituted hydrocarbylene. Typically, L is a halide, toluenesulfonate, or methylsulfonate; R₁₅ is hydrocarbylene or substituted hydrocarbylene having 1 to 18 carbons; and R₁₆ is selected from R₁₀OC(O)R₁₁—, R₁₀C(O)OR₁₁—, R₁₀OR₁₂OC(O)R₁₁—, R₁₃R₁₁—, and R₁₄R₁₁—, where R₁₀, R₁₁, R₁₂, R₁₃, and R₁₄, are as defined above. In one embodiment, R₁₅ is methylene or ethylene, and R₁₆ is —C(O)OCH₃, —C(O)OCH₂CH₃, phenyl, -2-(2-thienyl), or -2-(4-ethyl-4,5-dihydro-5-oxo-1H-tetrazol-1-yl)ethyl.

The alkylating agents may also comprise an electron deficient moiety to an electron withdrawing group such as carbonyl, nitrile, carbonyloxy, alkyl carbonate, and alkyl-alkoxy carbonate. Non-limiting specific examples of alkylating agents include methyl acrylate, ethyl acrylate, acrylic acid, acryronitrile, acrylamide, acrolein, phenylethyl halide, tolylate, mesylate, styrene, and substituted styrene. Alkylating agents comprising an electron deficient moiety may be depicted as follows:

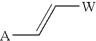

wherein A is hydrogen, hydrocarbyl, or substituted hydrocarbyl and W is hydrocarbyl, substituted hydrocarbyl, nitrile, or amide. In one example, A is hydrogen, linear or branched C₁₋₁₈alkyl, aryl, substituted aryl, alkylaryl, C₅₋₇ cycloalkyl or substituted C₅₋₇ cycloalkyl; and W is carboxylic acid, carboxylic acid ester, nitrile, amide, carbonyl, or aryl. In a preferred embodiment, A is hydrogen and W is a carboxylic acid ester or aryl.

Examples of the base used in the reaction of Scheme 1 include metal hydroxide, metal alkoxide, metal hydride, metal carbonate, metal hydrogen carbonate, amine, tetraalkyl ammonia hydroxide, and ammonia. Examples of metal alkoxides and metal hydrides include sodium, potassium, cesium, magnesium, aluminum alkoxides and hydrides and the like. Preferably, the base is triethylamine or tetraalkylamine hydroxide.

The solvent of Scheme 1 is an organic solvent. Typical solvents include dimethyl sulfoxide, ether, dichloromethane, chloroform, carbon tetrachloride, ethylene chloride, acetonitrile, toluene, ethylacetate, propylacetate, butylacetate, alcohol ethers, HMPA (hexamethyl phosphoramide), HMPT (hexamethyl phosphorimidic triamide), alkanols containing 1 to 18 carbon atoms, C₁₋₁₈hydrocarbyl, aryl-alcohol, and 5- to 7-membered heterocyclic alcohols comprising 1 to 5 heteroatoms selected from oxygen, sulfur, and nitrogen. In a preferred embodiment, the solvent is selected from the group consisting of acetonitrile and methanol.

In one embodiment, the reaction mixture comprises about 1 molar equivalent to about 5 molar equivalents of alkylating agent and about 1 molar equivalent to about 5 molar equivalents of base per molar equivalent of compound (II). For example, the reaction mixture may comprise about 1 to about 3 equivalents of an alkylating agent and about 1 to about 3 equivalents of base per molar equivalent of compound (II).

The solvent to compound (I) ratio on a volume to weight basis is about 1:2 to about 1:100; preferably, the solvent to compound ratio is about 1:4 to about 1:50.

In one embodiment, the temperature of the reaction mixture during the reaction ranges from about −10° C. to about 65° C. In one example of this embodiment, the reaction temperature ranges from about 10° C. to about 40° C. The reaction mixture is permitted to react up to a couple of days. In one example, the reaction is carried out up to about 24 hours. In another example, the reaction time is less than about 6 hours. In still another example, the reaction time is from about 0.5 hours to about 2 hours.

In one embodiment, methyl acrylate is added to compound (I) dispersed in methanol, followed by addition of triethylamine. The solution is then mixed for 1 hour. The resulting solid may be filtered off and the methanolic solution concentrated by vacuum to obtain compound (II). Compound (II) may be further purified through recrystallization with organic solvents, preparative chromatography or a combination of methods. Alternatively, Scheme 2 may proceed without isolation of compound (II).

Scheme 2, below, illustrates a second step in the process of the present invention wherein intermediate compound (III) is synthesized.

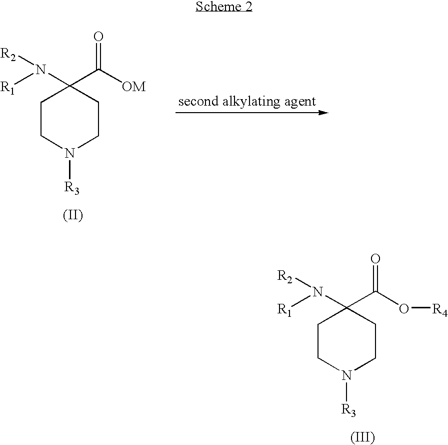

In Scheme 2, compound (II) is reacted with a second alkylating agent to form intermediate compound (III), wherein R₄ is hydrocarbyl or substituted hydrocarbyl. If compound (II) is not isolated during Scheme I, additional solvent is not required for Scheme 2. Alternatively, if compound (II) is isolated in Scheme I, a solvent is required.

Typically, R₄ is selected from the group consisting of C₁₋₁₈hydrocarbyl, R₁₇OR₁₈—, R₁₉R₁₈—, and R₂₀R₁₈—, wherein R₁₇ is hydrocarbyl or substituted hydrocarbyl, R₁₈ is hydrocarbylene or substituted hydrocarbylene, R₁₉ is aryl or substituted aryl, and R₂₀ is cycloalkyl, substituted cycloalkyl or heterocyclic. In one example of this embodiment, R₁₇ is substituted or unsubstituted alkyl, alkenyl, or alkynyl wherein the hydrocarbon chain contains 1 to 18 carbon atoms, R₁₈ is substituted or unsubstituted alkylene, alkenylene, or alkynylene wherein the hydrocarbon chain contains 1 to 18 carbon atoms, R₁₉ is aryl or substituted aryl, R₂₀ is C₃₋₆ cycloalkyl, substituted C₃₋₆ cycloalkyl or a 5- to 7-membered heterocyclic comprising 1 to 5 heteroatoms selected from oxygen, sulfur, and nitrogen. In another example of this embodiment, R₁₇ is substituted or unsubstituted alkyl, and R₁₈ is substituted or unsubstituted alkylene. In a preferred embodiment, R₃ is C₁₋₆alkyl; preferably, methyl, ethyl or propyl.

The second alkylating agent may be any of those alkylating agents described above for the first alkylating agent. In one embodiment, the second alkylating agent is a methylating, ethylating or propylating group. In one example of this embodiment, the second alkylating agent is a methylating agent selected from the group consisting of halomethane (e.g., iodomethane, bromomethane), dimethylsulfate, dimethylcarbonate, and chloromethane.

If a solvent is necessary, the solvent contained in this reaction mixture is an organic solvent. Typical solvents include dimethyl sulfoxide, ether, dichloromethane, chloroform, carbon tetrachloride, ethylene chloride, acetonitrile, toluene, ethylacetate, propylacetate, butylacetate, alcohol ethers, HMPA (hexamethyl phosphoramide), HMPT (hexamethyl phosphorimidic triamide), alkanols containing 1 to 18 carbon atoms, C₁₋₁₈hydrocarbyl, aryl-alcohol, and 5- to 7-membered heterocyclic alcohols comprising 1 to 5 heteroatoms selected from oxygen, sulfur, and nitrogen. In a preferred embodiment, the solvent is selected from the group consisting of acetonitrile and methanol.

Typically, the temperature of the reaction mixture during the reaction ranges from about 25° C. to about 80° C. For example, the temperature may range from about 50° C. to about 70° C. The reaction mixture is permitted to react up to a few days. In one example, the reaction occurs in less than about 24 hours. In another example, the reaction occurs in less than about 12 hours, preferably, from about 1 hour to about 4 hours.

If desired, a catalyst may be added to the reaction mixture. The catalyst is typically selected from the group commonly known as phase transfer catalysts. In one example, the catalyst is 18-crown-6, 15-crown-5, benzyl triethyl ammonium halide, benzyl tnbutyl ammonium halide, tetraalkyl ammonium halide. In one embodiment where a catalyst is added, the reaction mixture comprises about 1 molar equivalent to about 10 molar equivalents of the catalyst per molar equivalent of compound (III).

Depending on its physical properties, compound (III) may be purified and isolated by extraction, chromatography, distillation, filtration, or any combination of methods known in the art. In one embodiment, compound (III) is isolated by distillation of solvent, filtration or the addition of water, followed by solvent extraction of compound (III), and then drying by evaporation.

Scheme 3, below, illustrates a third step in the process of the present invention wherein the final opiate or opioid compound (IV) is synthesized.

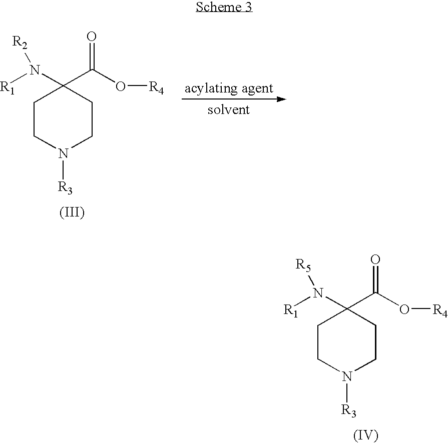

In Scheme 3, compound (III) is reacted with an acylating agent in a reaction mixture containing a solvent to form compound (IV), wherein R₅ is an acyl moiety corresponding to the acylating agent.

Typically, R₅ is —C(O)R₆ and R₆ is hydrocarbyl or substituted hydrocarbyl. In one example of this embodiment, the acylating agent is an acid halide, preferably a C₁₋₁₈ acid halide selected from alkyl acid halides and alkoxy-alkyl halides. Examples of acylating agents include, but are not limited to, acetyl chloride, acetic anhydride, propionyl chloride, propionic anhydride, methyl ketene, butanoyl chloride, alkyl acid cyanides, and the like. In one embodiment, the alkyl group comprises between 1 and about 18 carbon atoms. In another embodiment, the alkyl group comprises less than about 6 carbon atoms. For example, the alkyl group may comprise between 2 and 4 carbon atoms. Preferably the acylating agent is propionyl chloride or propionic anhydride.

The temperature of the reaction mixture ranges from about 20° C. to about 80° C. In one example of this embodiment, the reaction temperature ranges from about 40° C. to about 65° C. The reaction mixture is permitted to react from about 4 hours to about 18 hours. In one example, the reaction is carried out from about 4 hours to about 8 hours.

The solvent contained in the reaction mixture can be any solvent that is inert to the reaction occurring in Scheme 3. Examples of such solvents include, but are not limited to, acetonitrile, acetone, dichloromethane, chloroform, n,n-dimethylformamide, dimethylsulfoxide, ethylacetate, dichloroethane, aromatic hydrocarbons (e.g., benzene, toluene, and xylene), lower alkanols (e.g., methanol, ethanol, isopropanol, n-propanol, 1-butanol, tert-butanol), ketones (e.g., 4-methyl-2-pentanone), ethers (e.g., 1,4-dioxane, tetrahydrofuran (THF), 1,1-oxybisethane), nitrobenzene, and mixtures thereof. In one example, the reaction mixture comprises acetonitrile, chloroform, dichloromethane, or mixtures thereof. In another example, the reaction mixture comprises acetonitrile, chloroform, or a mixture thereof.

The reaction mixture optionally comprises an acid scavenger. Acid scavengers include metal hydrides, hydroxides, carbonates, bicarbonates, amines, and the like.

In one embodiment, the reaction mixture comprises about 1 molar equivalent to about 50 molar equivalents of acylating agent per molar equivalent of compound (III). Preferably, the reaction mixture comprises about 2 to about 5 molar equivalents of an acylating agent per molar equivalent of compound (III). The solvent to compound (III) ratio on a volume to weight basis is about 1:4 to about 1:50; preferably, the solvent to compound ratio is about 1:4 to about 1:25.

Compound (IV) is collected by filtration and drying. The product can be purified by methods known in the art including recrystallization, chromatography, and/or solvent extraction.

The overall process of the present invention for synthesizing opiate or opioid analgesics and anesthetics that incorporates the individual steps described above is illustrated in Scheme 4, below.

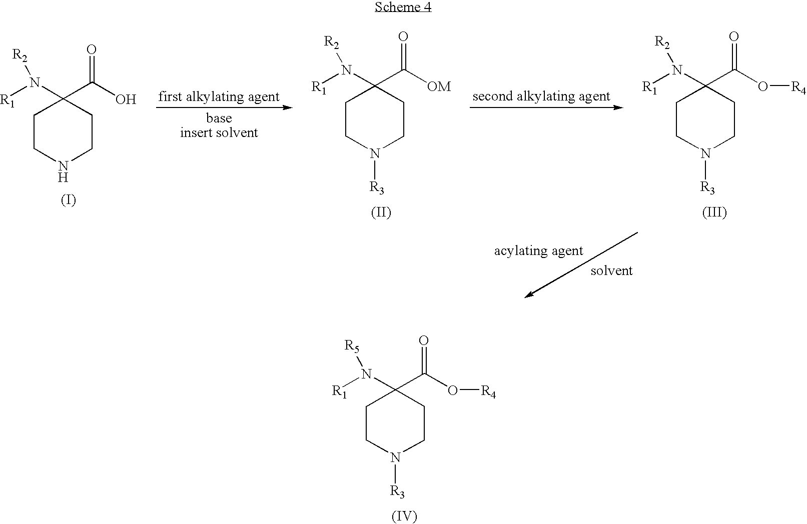

The process of the present invention described above reflects an improvement over the currently known synthetic reactions for producing analgesics or anesthetics by reducing the overall number of steps and eliminating the use of cyanide compounds.

In one embodiment of the present invention, a process for synthesizing remifentanil is provided. An illustration of this process is shown below in Scheme 5.

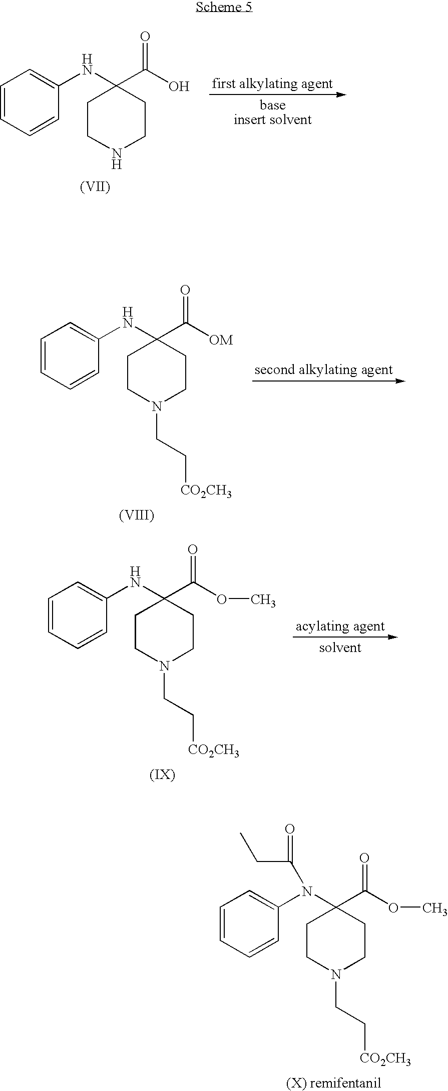

In Step 1, compound (VII), 4-(N-phenylamino)-4-carboxypiperidine, is reacted in a reaction mixture with a first alkylating agent and base to form compound (VIII).

In one embodiment, the reaction mixture comprises about 1 molar equivalent to about 5 molar equivalents of first alkylating agent and about 1 molar equivalent to about 5 molar equivalents of base per molar equivalent of compound (VII). Preferably, the reaction mixture comprises about 1 to about 3 molar equivalents of first alkylating agent and about 1 to about 3 molar equivalents of base per molar equivalent of compound (VII). The solvent to compound (VII) ratio on a weight to volume basis is about 1:2 to 1:100; preferably, the solvent to compound (VII) ratio is 1:4 to 1:30.

The temperature of the reaction mixture during the reaction ranges from about −10° C. to about 65° C. In another embodiment, the reaction temperature ranges from about 10° C. to about 40° C. The reaction mixture may be permitted to react up to a couple of days. In one example, the reaction is carried out in about 24 hours. In another example, the reaction time is less than about 12 hours. In still another example, the reaction time is from about 2 hours to about 6 hours.

Preferred solvents are selected from the group consisting of acetonitrile, chloroform, 1,2-dichloroethane, 1,1,2-trichloroethane, dichloromethane, carbon tetrachloride, and methanol.

Typically, the base used in the reaction is a strong base. For purposes of this invention, a strong base has a pH of 10 or higher. Preferably, the base is a hydroxide, such as sodium hydroxide or tetraalkyl ammonium hydroxide.

In one embodiment, methyl acrylate is added to compound (VII) dispersed in methanol. Triethylamine is added and mixed for 1 hour. The resulting solid is filtered off and the methanolic solution concentrated by vacuum to obtain compound (VIII). Compound (VIII) may be further purified through recrystallization with organic solvents, preparative chromatography, or a combination of methods.

In one embodiment, the reaction mixture comprises about 2 molar equivalents to about 100 molar equivalents of methanol per molar equivalent of compound (VIII). In one example of this embodiment, the reaction mixture comprises about 4 molar equivalents to about 50 molar equivalents of methanol per molar equivalent of compound (VIII).

Typically, the temperature of the reaction mixture during the reaction ranges from about 25° C. to about 80° C. For example, the reaction temperature may range from about 50° C. to about 70° C. The reaction mixture may be permitted to react up to several days. In one example, the mixture is reacted from about 8 to about 100 hours. Preferably, the reaction time is from about 24 hours to about 60 hours.

If desired, a catalyst may be added to the reaction mixture. The catalyst is typically selected from the group commonly known as phase transfer catalysts. In one example, the catalyst is 18-crown-6, 15-crown-5, benzyl triethyl ammonium halide, benzyl tributyl ammonium halide, tetraalkyl ammonium halide. In one embodiment where a catalyst is added, the reaction mixture comprises about 1 molar equivalent to about 10 molar equivalents of the catalyst per molar equivalent of compound (VIII).

In Step 2, compound (VIII) is reacted with a second alkylating agent to form compound (IX). This reaction may occur either with or without isolation of compound (VIII) from Step 1. In one embodiment, the second alkylating agent is a methylating agent such as halomethane (e.g., iodomethane, bromomethane), dimethylsulfate, dimethylcarbonate, and chloromethane. In one example of this embodiment, the second alkylating agent is dimethylsulfate.

In one embodiment of Step 2, the temperature of the reaction mixture during the reaction ranges from about 25° C. to about 80° C. For example, the temperature may range from about 35° C. to about 70° C. Typically, the reaction mixture is permitted to react for less than about 12 hours, preferably, from about 1 hour to about 4 hours.

Depending on its physical properties, compound (IX) may be purified and isolated by extraction, chromatography, distillation, or any combination of methods known in the art. In one embodiment, compound (IX) is isolated by the addition of water, followed by solvent extraction of compound (IX), and finally drying by evaporation. In one example, compound (IX) can be purified and isolated by adding water and toluene to the solution, which has been concentrated to dryness under vacuum. The water is then separated and hexane added to crystallize the product. The solution can then be filtered off and the solid washed with hexane. Finally, the solid can be dried in a vacuum oven to obtain compound (IX).

In Step 3, compound (IX) is reacted with an acylating agent in a reaction mixture containing a solvent to form remifentanil (compound (X)). In one embodiment, the acylating agent is propionyl chloride or propionic anhydride.

The temperature of the reaction mixture ranges from about 20° C. to about 80° C. In one example of this embodiment, the reaction temperature ranges from about 40° C. to about 65° C. The reaction mixture is permitted to react from about 4 hours to about 18 hours, preferably from about 4 hours to about 8 hours.

The solvent contained in the reaction mixture can be any solvent that is inert to the reaction occurring in Step 3. Examples of such solvents include, but are not limited to, acetonitrile, acetone, dichloromethane, chloroform, n,n-dimethylformamide, dimethylsulfoxide, ethylacetate, dichloroethane, aromatic hydrocarbons (e.g., benzene, toluene, and xylene), ketones (e.g., 4-methyl-2-pentanone), ethers (e.g., 1,4-dioxane, tetrahydrofuran (THF), 1,1-oxybisethane), nitrobenzene, and mixtures thereof. In one example, the reaction mixture comprises acetonitrile.

Typically, the reaction mixture comprises about 1 molar equivalent to about 50 molar equivalents of acylating agent per molar equivalent of compound (IX). For example, the reaction mixture may comprise about 2 to about 5 molar equivalents of an acylating agent per molar equivalent of compound (IX). The solvent to compound (IX) ratio on a volume to weight basis is about 1:4 to about 1:25; preferably, the solvent to compound ratio is 1:4 to 1:15.

Remifentanil is collected by filtration and drying. The product can be purified by recrystallization, solvent extraction, or any other methods or combination of methods known in the art.

In another embodiment, Scheme 5 is modified to prepare carfentanil. The method of preparing carfentanil is nearly identical to that of remifentanil with the exception of Step 3, wherein the alkylating compound used to produce carfentanil is typically styrene or phenylethyl halide.

### Examples

The following examples are provided in order to more fully illustrate the present invention.

### Example 1

### Reaction Steps 1 and 2

(A) 4-(N-aminophenyl)-4-carboxypiperidine (1 g) and tetramethylammonium hydroxide pentahydrate (0.8 g) were stirred in methanol (20 ml) until all solid was dissolved, followed by the addition of 0.5 ml of methyl acrylate. The solution was stirred at mom temperature for 1 hour. The solution was concentrated to dryness under vacuum to obtain an oil. Acetonitrile (20 ml) was added followed by iodomethane (0.3 ml). The solution was stirred at room temperature overnight. The solid was filtered and the solution was concentrated to dryness to obtain a clear oil. 10 ml of 1 N HCl was added to dissolve the solid, then the pH of the solution was adjusted to about 9 with concentrated ammonium hydroxide. The solution was stirred at room temperature until the product crystallized. The solution was filtered and washed with water to obtain methyl 3-(4-N-aminophenyl-4-carbomethoxy-piperidino)propionate as white solid.

(B) 4-(N-aminophenyl)-4-carboxypiperidine (20 g), methanol (150 ml), sodium methoxide 25% solution (20 g), and methyl acrylate (8.8 g) were combined and stirred for about one hour. HPLC indicated that no 4-(N-aminophenyl)-4-carboxypiperidine remained. The solution was filtered to remove undissolved solids. The cake was then washed with methanol. The filtrate and wash were combined and concentrated hydrochloric acid (9.1 g) was added. Most of the methanol was removed by vacuum. Water (150 ml) was added to the residue and stirred for approximately 15 minutes. The solids were filtered and dried. Yield 23 g. HPLC indicated 100% area MW 306.

(C) 4-(N-aminophenyl)-4-carboxypiperidine (1 g) and tetramethylammonium hydroxide pentahydrate (0.8 g) were stirred in acetonitdle (20 ml) at 40° C. until all solid was dissolved, followed by the addition of 0.5 ml of methyl acrylate. The solution was stirred at room temperature for 3 hours. Iodomethane (0.3 ml) was added to the solution and stirred at room temperature overnight. The solid was filtered and the solution was concentrated to dryness to obtain a clear oil. 10 ml of 1 N HCl was added to dissolve the solid, then the pH of the solution was adjusted to about 9 with concentrated ammonium hydroxide. The solution was stirred at room temperature until the product crystallized. The solution was filtered and washed with water to obtain methyl 3-(4-N-aminophenyl-4-carbomethoxy-piperdino) propionate as white solid.

### Example 2

### Reaction Step 3

Methyl 3-(4-N-aminophenyl4-carbomethoxy-pipeddino) propionate (10 g), 10 ml of propionyl chloride and 100 ml of chloroform were stirred at reflux (63° C.) for 12 hours. The solution was concentrated to dryness under vacuum. Acetonitrile was added to disperse the solid. The solution was filtered to obtain remifentanil hydrochloride as white solid.

### Example 3

### Preparation of Remifentanil HCl

4-(N-aminophenyl)-4-carboxypiperidine (10 g) and tetramethyl ammonium hydroxide pentahydrate (9 g) were charged in 200 ml acetonitrile (1 part 4-(N-aminophenyl)-4-carboxypiperidine to 120 parts acetonitrile). The solution was stirred at 40° C. for 1 hour until most of the solid was dissolved. Methyl acrylate (5 ml) was added and the solution stirred at 40° C. for about an hour. The presence of the intermediate compound, methyl-3-(4-N-aminophenyl-4-carboxcylic acid-piperidino)proppionate, was verified by liquid chromatography.

After the first alkylation was complete, dimethyl sulfate (5 ml) was added to the solution and stirred for 4 hours at 40° C. The solution was then concentrated to dryness under vacuum at 40° C. Water (200 ml) and toluene (400 ml) were added, followed by separation of the water. Hexane (400 ml) was then added to crystallize the product. The solution was filtered off and the solid was washed with hexane. The solid was dried at 60° C. in a vacuum oven for 4 to 12 hours to obtain the white solid, the second intermediate compound, 3-(4-N-aminophenyl-4-carbomethoxy-pipeddino)propionate.

The second intermediate (10 g) was dissolved in chloroform (100 ml) followed by the addition of propionyl chloride (10 ml). The solution was stirred at 60° C. for 8 hours then cooled to room temperature. Methanol (10 ml) was added and the solution stirred for 15 minutes at room temperature to destroy excess propionyl chloride. The solution was then concentrated to either an oil or solid. Acetone (100 ml) was added and the solution stirred at room temperature for 30 minutes to crystallize the product. The solvent was then filtered off and washed with acetone (50 ml) to obtain crude remifentanil HCl as white solid.

### ABBREVIATIONS AND DEFINITIONS

The term “acyl” denotes a radical provided by the residue after removal of hydroxyl from an organic acid, for example, COOH of an organic carboxylic acid, e.g., RC(O)—, wherein R is R₂₁, R₂₁O—, R₂₁R₂₂N—, or R₂₂S—, R₂₁ is hydrocarbyl, heterosubstituted hydrocarbyl, or heterocyclo and R₂₂ is hydrogen, hydrocarbyl or substituted hydrocarbyl. Examples of such acyl radicals include alkanoyl and aroyl radicals. Examples of lower alkanoyl radicals include formyl, acetyl, propionyl, butyryl, isobutyryl, valeryl, isovaleryl, pivaloyl, hexanoyl, and trifluoroacetyl.

The term “alkyl” denotes a linear or branched radical of one to about twenty carbon atoms or, preferably, one to about 12 carbon atoms. More preferred alkyl radicals are “lower alkyl” radicals having one to about six carbon atoms. Examples of alkyl radicals include methyl, ethyl, propyl, isopropyl, butyl, isobutyl, tert-butyl, sec-butyl, pentyl, hexyl, and the like.

The term “alkenyl” denotes a linear or branched radical having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl. The terms “alkenyl” and “lower alkenyl” also are radicals having “cis” and “trans” orientations, or alternatively, “E” and “Z” orientations.

The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” denote linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” denotes an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “amino” as used herein alone or as part of another group denotes the moiety —NR₂₃R₂₄ wherein R₂₃ and R₂₄ are hydrocarbyl, substituted hydrocarbyl or heterocyclo.

The terms “halide,” “halogen,” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties comprise 1 to 18 carbon atoms. They may be straight or branched chain or cyclic and include methyl, ethyl, propyl, isopropyl, allyl, benzyl, hexyl and the like.

The term “hydrocarbylene” as used herein describes radicals joined at two ends thereof to other radicals in an organic compound, and which consist exclusively of the elements carbon and hydrogen. These moieties include alkylene, alkenylene, alkynylene, and arylene moieties. These moieties also include alkylene, alkenylene, alkynylene, and arylene moieties substituted with other aliphatic or cyclic hydrocarbon groups, such as alkaryl, alkenaryl and alkynaryl. Unless otherwise indicated, these moieties preferably comprise 1 to 18 carbon atoms. They may be straight or branched chain or cyclic and include methylene, ethylene, propylene, isopropylene, allylene, benzylene, hexylene and the like.

The “substituted hydrocarbyl” and “substituted hydrocarbylene” moieties described herein are hydrocarbyl and hydrocarbylene moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, keto, acyl, acyloxy, nitro, tertiaryamino, amido, nitro, cyano, ketals, acetals, esters and ethers.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that the several objects of the invention are achieved and other advantageous results attained.

As various changes could be made in the above methods and products without departing from the scope of the invention, it is intended that all matter contained in the above description and shown in any accompanying drawings shall be interpreted as illustrative and not in a limiting sense.

1-29. (canceled)

30. A process for the preparation of an opiate or opioid analgesic or anesthetic, the process comprising:
alkylating with a first alkylating agent a compound (I) having the formula: 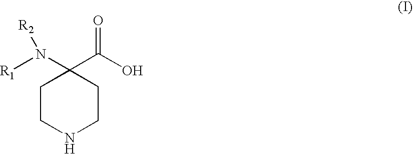
wherein R₁ and R₂ are independently hydrogen, hydrocarbyl or substituted hydrocarbyl in the presence of a solvent and a base to form intermediate compound (II): 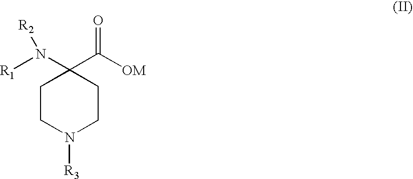
wherein M is hydrogen, a cation, amino, or substituted amino and R₃ is hydrocarbyl or substituted hydrocarbyl;
alkylating the compound (II) with a second alkylating agent to form an intermediate compound (III): 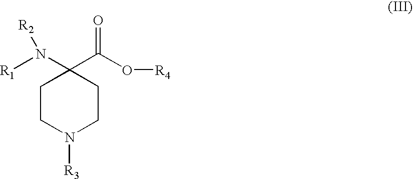
wherein R₄ is hydrocarbyl or substituted hydrocarbyl; and
acylating the compound (III) with an acylating agent in the presence of a solvent to form the opiate or opioid, compound (IV): 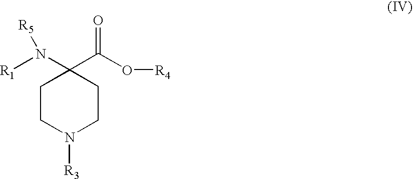
wherein R₅ is —C(O)R₆ and R₆ is hydrocarbyl or substituted hydrocarbyl.

31. The process of claim 1 wherein
R₁ and R₂ are independently H, aryl, substituted aryl, C₁₋₁₈alkyl, cycloalkyl, substituted cycloalkyl, heterocyclic, R₇OR₈— or R₉R₈—;
R₇ is hydrocarbyl or substituted hydrocarbyl;
R₈ is hydrocarbylene or substituted hydrocarbylene; and
R₉ is cycloalkyl, substituted cycloalkyl, or heterocyclic.

32. The process of claim 2 wherein R₁ is phenyl and R₂ is H.

33. The process of claim 32 wherein R₃ is selected from the group consisting of aryl, substituted aryl, aralkyl, C₁₋₁₈alkyl, R₁₀OC(O)R₁₁—, R₁₀OC(O)OR₁₁—, R₁₀OR₁₂OC(O)R₁₁—, R₁₃R₁₁—, and R₁₄R₁₁—;
R₁₀ is hydrocarbyl or substituted hydrocarbyl;
R₁₁ and R₁₂ are independently hydrocarbylene or substituted hydrocarbylene;
R₁₃ is cycloalkyl or substituted cycloalkyl; and
R₁₄ is heterocyclic.

34. The process of claim 33 wherein R₁₀ is alkyl, alkoxy, alkenyl, aryl, aralkyl or alkenyloxy;
R₁₁, and R₁₂ are independently alkylene, alkyleneoxy, alkenylene, arylene, aralkylene or alkenyleneoxy;
R₁₃ is a 5- to 7-membered cycloalkyl; and
R₁₄ is a 5- to 7-membered heterocyclic.

35. The process of claim 34 wherein R₁₀ is linear or branched C₁₋₁₈alkyl, C₁₋₁₈alkoxy, C₂₋₁₈alkenyl, or C₂₋₁₈alkenyloxy; R₁₁ and R₁₂ are independently linear or branched C₁₋₁₈alkylene, C₁₋₁₈alkyleneoxy, C₂₋₁₈alkenylene, or C₂₋₁₈alkenyleneoxy;
R₁₃ is C₅₋₇cycloalkyl; and
R₁₄ is a 5- to 7-membered heterocyclic comprising 1 to 5 heteroatoms selected from oxygen, sulfur, and nitrogen.

36. The process of claim 35 wherein M is a cation, amino, or substituted amino.

37. The process of claim 36 wherein M is hydrogen, a cation selected from the group consisting of sodium, potassium, and lithium, or tetraalkylamino.

38. The process of claim 37, wherein said solvent is selected from the group consisting of water, acetonitrile, acetone, dichloromethane, chloroform, n,n-dimethylformamide, dimethylsulfoxide, ethylacetate, dichloroethane, triethylamine, benzene, toluene, xylene, methanol, ethanol, isopropanol, n-propanol, 1-butanol, tert-butanol, 4-methyl-isopropanol, 1,4-dioxane, tetrahydrofuran (THF), 1,1-oxybisethane, nitrobenzene, and mixtures thereof.

39. The process of claim 38 wherein the first alkylating agent is selected from the group consisting of methyl acrylate, ethyl acrylate, acrylic acid, acrylonitrile, acrylamide, acrolein, a phenylethyl halide, tolylate, mesylate, styrene, and a substituted styrene.

40. The process of claim 39 wherein the base is selected from the group consisting of a metal hydroxide, a metal alkoxide, a metal hydride, a metal carbonate, a metal hydrogen carbonate, an amine, a quaternary alkyl ammonia hydroxide, and ammonia.

41. The process of claim 40 wherein the second alkylating agent is selected from the group consisting of an alkyl acrylate, a haloalkyl, a benzyl halide, benzyl tosylate, benzyl OMS, phenyethyl OMS, a phenylethyl halide, thienylethyl OMS, thienylethyl alkyl sulfate, alkyl carbonate, OTS, and a thienylethyl halide.

42. The process of claim 41 wherein the second alkylating agent is selected from the group consisting of halomethane, dimethylsulfate, and dimethylcarbonate.

43. The process of claim 42 wherein R₄ is selected from the group consisting of C₁₋₁₈hydrocarbyl, R₁₇OR₁₈—, R₁₉R₁₈—, and R₂₀R₁₈—;
R₁₇ is hydrocarbyl or substituted hydrocarbyl;
R₁₈ is hydrocarbylene or substituted hydrocarbylene;
R₁₉ is aryl or substituted aryl; and
R₂₀ is cycloalkyl, substituted cycloalkyl or heterocyclic,

44. The process of claim 43 wherein R₄ is C₁₋₆alkyl.

45. The process of claim 44 wherein the compound (III) is formed in the presence of a phase transfer catalyst.

46. The process of claim 45 wherein the acylating agent is selected from the group consisting of acetyl chloride, acetic anhydride, ethanoyl chloride, propionyl chloride, propionic anhydride, methyl ketene, butanoyl chloride, and an alkyl acid cyanide.

47. The process of claim 46 wherein compound (IV) is remifentanil or carfentanil.

48. The process of claim 47 wherein compound (II) is not isolated.

49. The process of claim 47 wherein compound (II) is isolated.
